(19) United States
(12) Patent Application Publication
(10) Pub. No.: US 2021/0147772 A1
(43) Pub. Date: May 20, 2021

(54) APPARATUS AND METHOD FOR RAPID PHYTOREMEDIATION REACTION

(71) Applicants: ARTHUR JOHN FROESE, Calgary (CA); DAVE GERMANIUK, Sexsmith (CA)

(72) Inventors: ARTHUR JOHN FROESE, Calgary (CA); DAVE GERMANIUK, Sexsmith (CA)

(21) Appl. No.: 16/950,299

(22) Filed: Nov. 17, 2020

Related U.S. Application Data
(60) Provisional application No. 62/937,565, filed on Nov. 19, 2019.

(51) Int. Cl. C12M 1/00

(52) U.S. Cl. CPC C12M 21/02; C12M 29/06; C12M 23/34; C12M 23/02

(57) ABSTRACT

A rapid phytoremediation reactor apparatus includes a tube, and the tube's interior has a rough texture configured for supporting growth of algae. A method for cultivating algae includes flowing a liquid through a tube with periphyton growing on an inner surface of the tube, the tube being penetrable to light; illuminating the tube to grow the periphyton on the inner surface of the tube; and harvesting the periphyton.

### BENEFIT OF EARLIER APPLICATIONS

This application claims priority from U.S. provisional application 62/937,565 filed Nov. 19, 2019.

### TECHNICAL FIELD

The present invention relates to phytoremediation and, in particular, algal scrubbing.

### BACKGROUND

Periphyton may be used to remove pollutants or nutrients from water or other fluid sources. For example, periphyton may be used to remove CO₂ introduced by combustion processes in order to remove or “scrub” gas and liquid pollution from a fluid source. Periphyton can be deployed to uptake nutrients from industrial process streams, in order to reduce emissions of such pollutants. As such, there is a market demand for periphyton technologies and algal technologies. Legal, regulatory, and environmental factors may increase the demand for emissions reduction and therefore periphyton, including benthic algae.

Industrial processes, including municipal water treatment, have a detrimental effect on water bodies due to their biological oxygen demand from phosphate and nitrogen, creating algal growth in the streams. When decomposed, these algal growths have an oxygen demand on the body of water, and thereby harm the downstream ecology. In addition to pollutant streams from industry, water runoff from farming, as well as niche industries such as shrimp and fish farming, create harmful effluent streams that impact the downstream aquatic life. Current technology for remediation includes mechanical aeration, water volume turnover/dilution, and chemical treatment.

Chemical treatment and neutralization both attempt to remove direct toxicity of streams. Chemical treatment and neutralization introduce indirect consequences through increased biological oxygen demand in the streams, and reduced or eliminated dissolved oxygen as compared to the diverted stream. Typical treatments of streams include flocculants, coagulants and biocides (for example, chlorine). These chemicals will be present in post-treatment effluent. There is therefore a need for a treatment apparatus and method that does not use such chemicals.

In the example of fish farming, the living area of the fish can either be (i) enclosed, or (ii) in a natural habitat contained with barriers. Higher concentration of fish produces a higher concentration of pollutants, which harms the surrounding ecological area. Using a rapid phytoremediation reactor (RPR) improves the efficiency of the ecosystem's pollutant reduction process and reduces the ecological harm generated by the fish farm.

Periphyton is a mix of benthic algae and other organisms that may be grown under artificial or natural light. The use of artificial light rather than natural light may promote growth of higher oxygen-producing species of algae, which may remove pollutants more efficiently. Periphyton is type of algae that naturally grows on hard bottoms of bodies of water that receive light. Compared to periphyton, planktonic algae are more capable of growing elsewhere in a body of water, that is, not necessarily on a surface, but anywhere in the illuminated body of water.

Algal turf is “short” or “low-lying” algae comprised of one or more species, including, for example, periphyton, which is often filamentous benthic algae and cyanobacteria, among other components. Algal turf scrubbing has been studied extensively and is believed, compared to other producers, to be able to produce greater algal yields in shorter amounts of time, and correspondingly to have a more efficient capacity for reduction of pollutants. The biomass can be used as animal food, feedstock for biofuel, and in pharmaceutical products. Periphyton is commonly grown using open water sources on flat surfaces. These methods are prone to contamination and occupy a large and inefficient footprint.

Typical algae production and pollutant fluid remediation (also known as “turf scrubbing”) involves providing a flat-surfaced, open body of water, for example a pond, illuminating the pond to grow periphyton on a substrate of the pond, and using a wave action or flow channel to promote contact between the periphyton and the pollutants identified for reduction. This two dimensional substrate is not an efficient use of space. That is, only a two-dimensional substrate or layer of the pond is used to grow periphyton. Commercialization of such technology requires a large area of land. When relying on natural light, little or no periphyton is produced during the night. These are problems with the prior art.

Compared to periphyton, planktonic algae cannot be grown as quickly or densely; can be less efficient at photosynthesis; and are more difficult to harvest due to requiring fine filtration or centrifugal separation, whereas periphyton are removed in larger pieces. Therefore, planktonic algae are relatively less desirable as a pollutant treatment and biomass product.

### SUMMARY OF INVENTION

In accordance with a broad aspect of the present invention, there is provided a rapid phytoremediation reactor apparatus comprising: a tube, the tube's interior having a rough texture configured for supporting growth of algae.

In accordance with another broad aspect of the present invention, there is provided a method for cultivating algae, comprising: flowing a liquid through a tube with periphyton growing on an inner surface of the tube, the tube being penetrable to light; illuminating the tube to grow the periphyton on the inner surface of the tube; and harvesting the periphyton.

It is to be understood that other aspects of the present invention will become readily apparent to those skilled in the art from the following detailed description, wherein various embodiments of the invention are shown and described by way of illustration. As will be realized, the invention is capable of other and different embodiments and its several details are capable of modification in various other respects, all within the present invention. Furthermore, the various embodiments described may be combined, mutatis mutandis, with other embodiments described herein. Accordingly, the drawings and detailed description are to be regarded as illustrative in nature and not as restrictive.

### BRIEF DESCRIPTION OF THE DRAWINGS

Referring to the drawings, several aspects of the present invention are illustrated by way of example, and not by way of limitation, in detail in the figures, wherein:

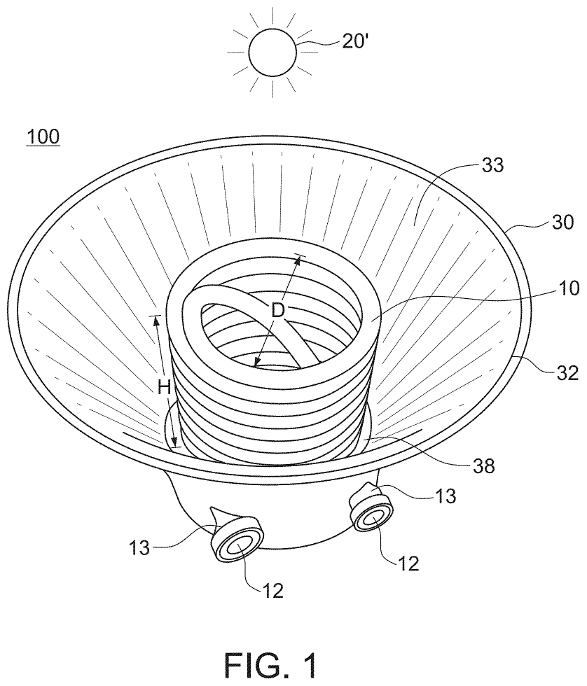

(a) FIG. 1 is a top perspective view of an embodiment of a reactor including a helically wound tube and a conical side wall with reflective coating;

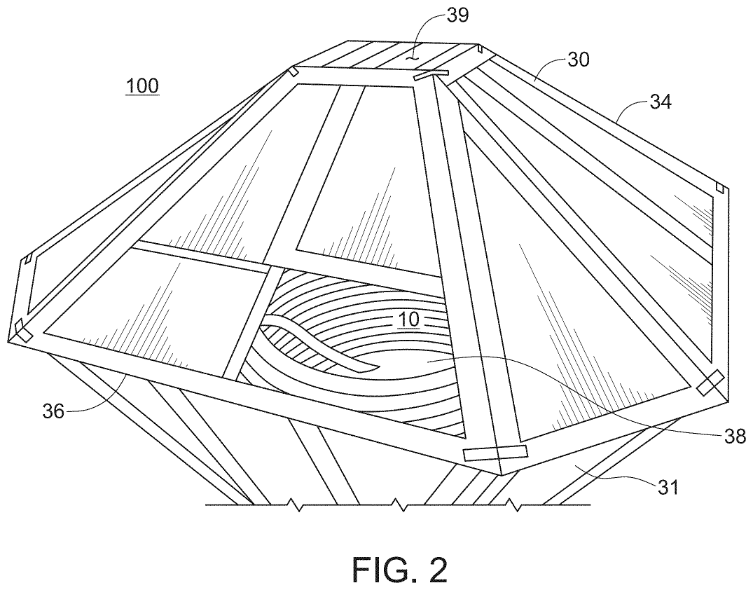

(b) FIG. 2 is a top perspective view of another embodiment of a reactor including a helically wound tube, and pyramidal side wall and lid;

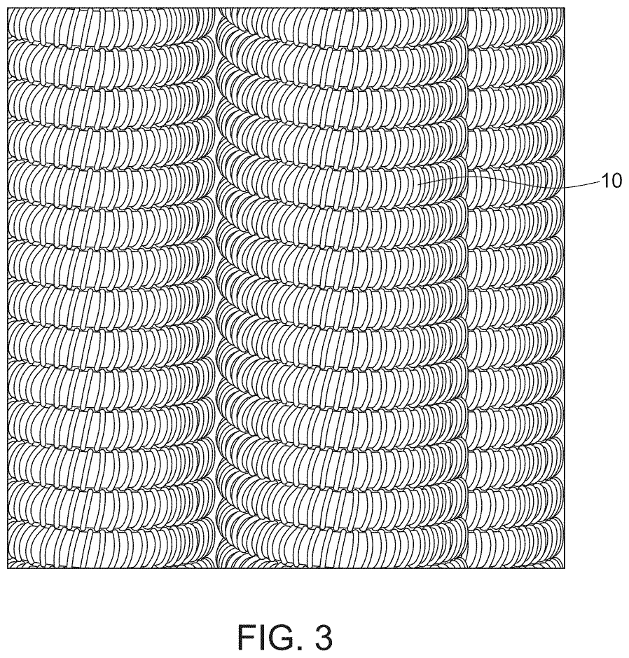

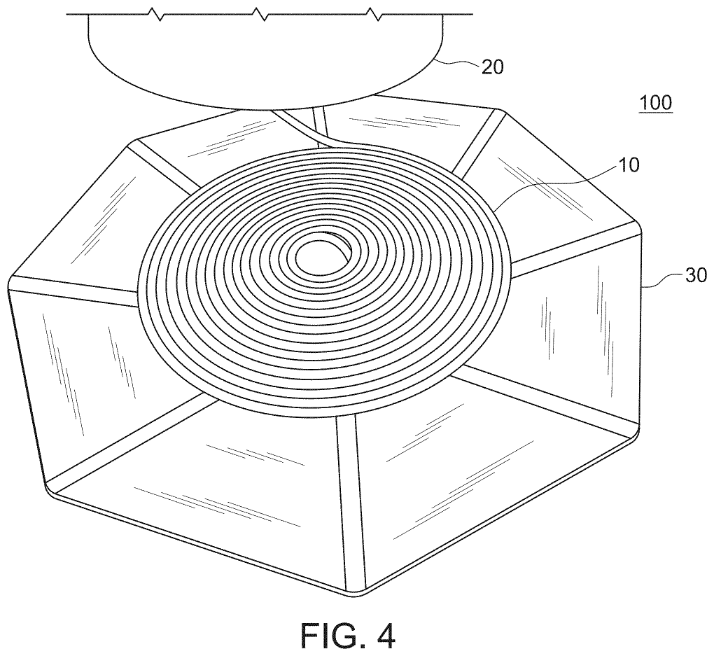

(c) FIG. 3 is a front perspective view a helically wound tube according to another embodiment;

(d) FIG. 4 is a top perspective view of another embodiment of a tube wound in a spiral on a plane;

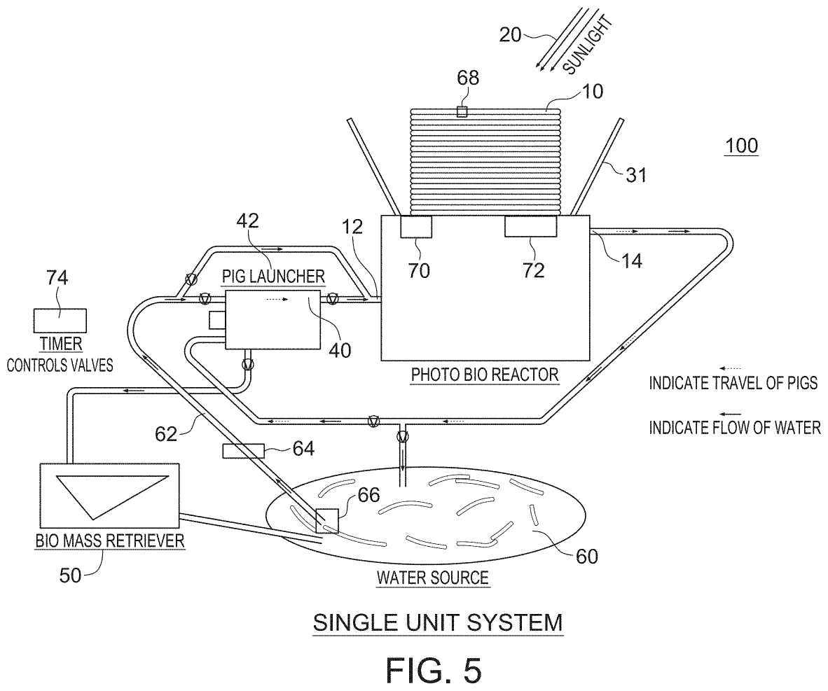

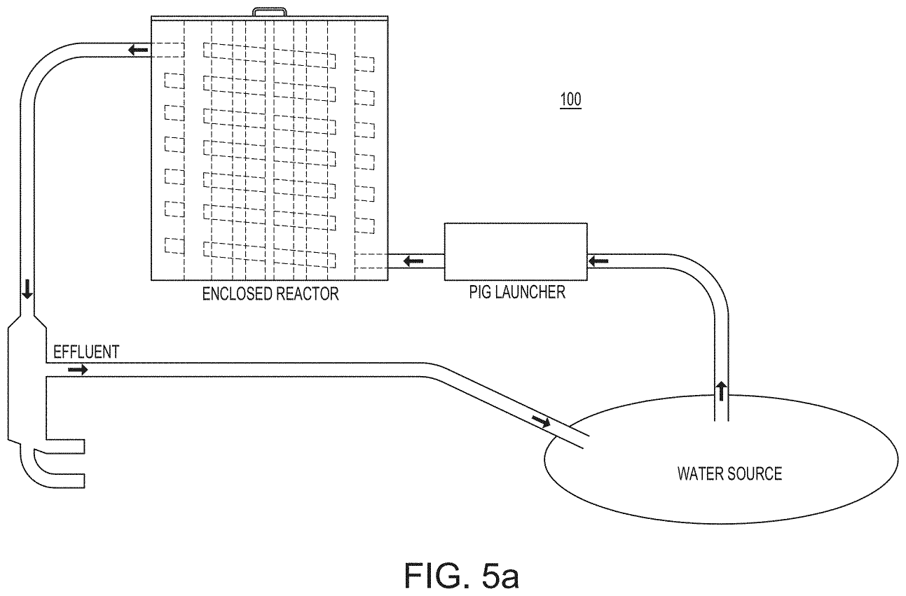

(e) FIG. 5 is a diagram of another embodiment of a reactor including a pig launcher, biomass retriever, reservoir, and other optional elements;

(f) FIG. 5a is a diagram of another embodiment of a reactor including a pig launcher;

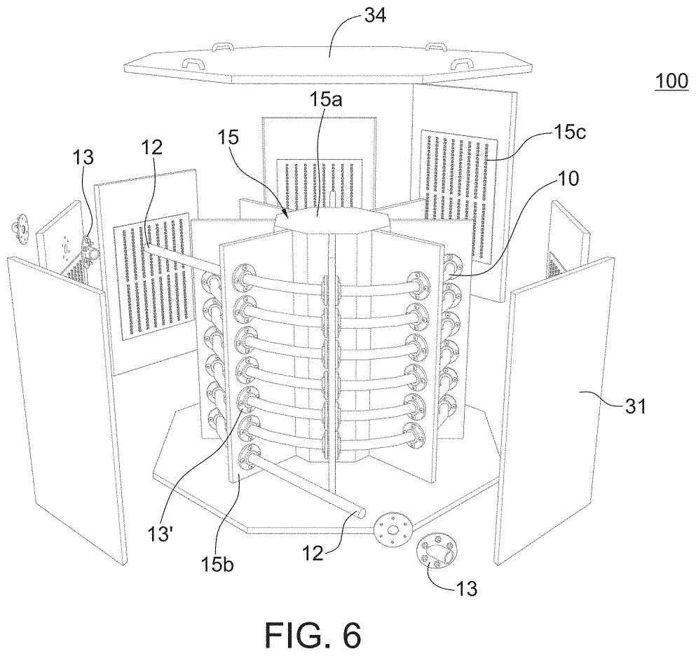

(g) FIG. 6 is a partly exploded side perspective view of another embodiment of a reactor including a support structure with support arms;

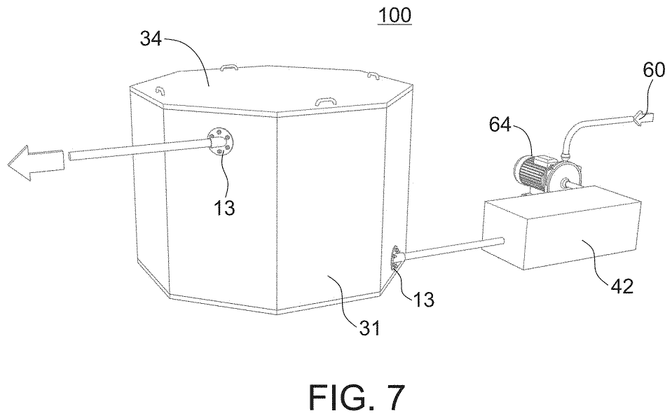

(h) FIG. 7 is a side perspective view of another embodiment of a reactor including a reservoir, pump, and pig launcher;

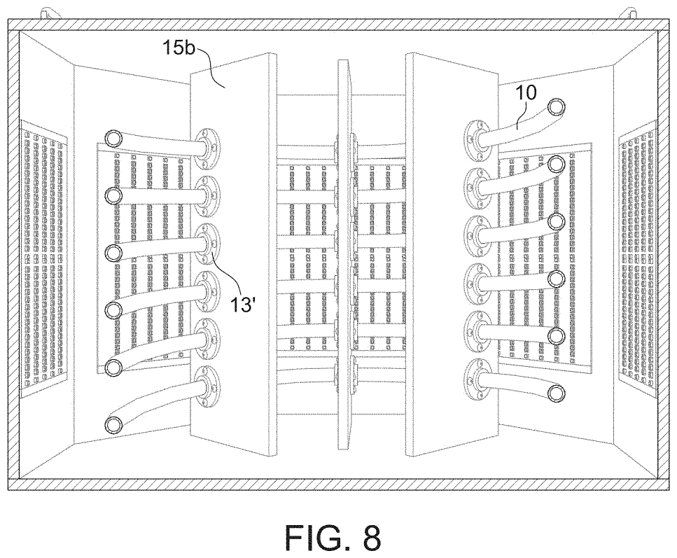

(i) FIG. 8 is a cross sectional view of part of the reactor of FIG. 7 along line 8-8 in FIG. 7; and

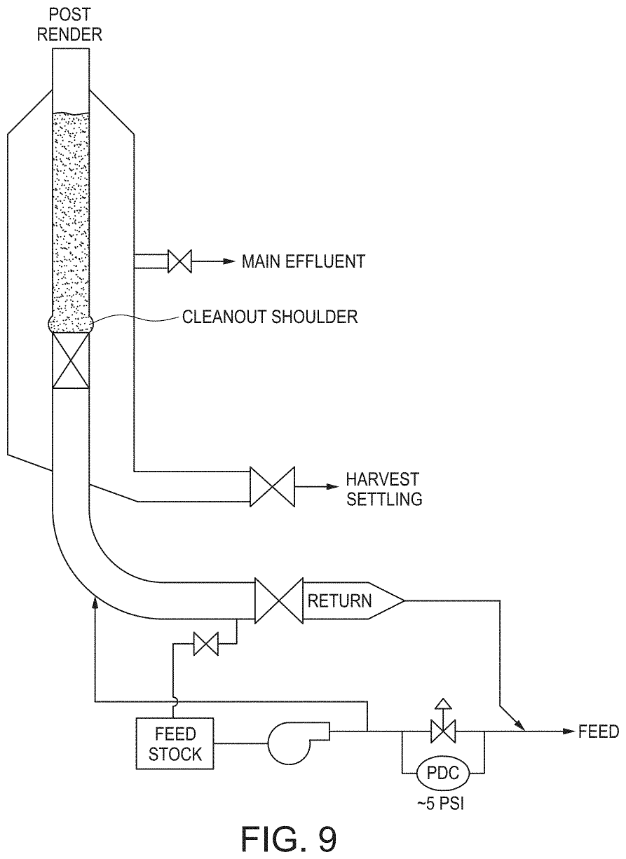

(j) FIG. 9 is a diagram of a biomass retriever according to another embodiment of a reactor.

### DETAILED DESCRIPTION OF EMBODIMENTS

The detailed description set forth below in connection with the appended drawings is intended as a description of various embodiments of the present invention and is not intended to represent the only embodiments contemplated by the inventor. The detailed description includes specific details for the purpose of providing a comprehensive understanding of the present invention. However, it will be apparent to those skilled in the art that the present invention may be practiced without these specific details. While this paper refers to periphyton, it is to be understood that other organisms, including other types of algae, may be produced using the present invention. While this paper refers to pollutants, it is to be understood that in certain circumstances, such substances may also be nutrients. That is, a “pollutant” may be a contaminant in some circumstances, and may support growth and life in other circumstances.

### Apparatus and Method for Growing Periphyton and Treating Source Fluid in an Illuminated Tube

The present invention provides a method and rapid phytoremediation reactor apparatus for fluid purification using periphyton production in an illuminated tube. Periphyton can grow attached to a surface. In use, a source fluid including water and periphyton microorganisms may be introduced into the tube to create an algal growth in the tube. The tube, which is transparent, is illuminated, either by natural or artificial light. Light promotes growth of periphyton. Periphyton may grow on the interior of the tube. Fluid may be treated by passing through the tube, the fluid being treated by coming into contact with periphyton. Periphyton may be trimmed to control growth thereof and the harvested periphyton may be used outside the apparatus.

Periphyton being grown in tubes provides several advantages over being grown in an open, flat body of water such as a pond, including: lower rates of contamination compared to methods involving an open water source; and, as the tube has a greater surface area per cubic metre of space used for the apparatus, increased yield of periphyton and more efficient use of light.

The reactor may be used for various purposes, including airborne contaminant scrubbing (including H2S and CO2), water oxygenation, and pollutant removal from pollutant streams and stagnant bodies. The harvested periphyton can be used for food, pharmaceuticals, seed, a nutrient source, and fertilizer, among other uses.

Referring to the Figs., a rapid phytoremediation reactor apparatus 100 may include a tube 10 and a housing 30. In the illustrated embodiment, the tube is coiled, and is illuminated both directly by natural light 20′, and indirectly by reflecting the natural light off of a reflective coating 33 on the housing.

### Tube

The tube may be substantially continuous. In other words, the tube may form a closed loop. The tube may have a substantially constant wall thickness. An interior of the tube may have a rough surface. For example, the interior of the tube may have asperities, scratches, grooves, bumps, ribs, indents, other textures, or a combination of textures. Relative to a smooth interior, a textured interior increases the surface area of the inner wall of the tube, thereby increasing the surface area upon which periphyton may grow compared to a reactor with a tube of the same length and a smooth interior. Periphyton may grow faster on rough surface compared to a smooth surface. The rough interior may also promote turbulent flow of fluid through the tube, which can improve the yield of periphyton. Erratic flow may allow dissolved contaminants to be mixed with the source fluid, making a greater distribution of contaminants available to the periphyton. Laminar flow may hold periphyton' filaments into an immobile flattened position, thereby hindering contact with pollutants. Turbulent flow encourages periphyton' exposure to water and light. Further possible advantages of the optional rough surface of the interior of the tube are described at [0037] and elsewhere in this paper.

In one embodiment, the tube may be coiled vertically, as illustrated in FIGS. 1-3, forming a cylinder shape. The tube may be helically wound about a vertical axis. The tube may be coiled on itself, with each turn in the coil set on top of the last, thereby defining an inner diameter and an outer diameter.

Coiling the tube may improve turbulent flow within the tube, thereby improving periphyton yield. A vertically extending coil also permits evacuation of gas. When coiled, the tube may form a cylinder shape with diameter D (FIG. 1). The tube may extend upwards to height H selected to accommodate headroom, lighting panel maximums, or remediation requirements. Remediation requirements may include, for example, concentration of periphyton to water, temperature, or other environmental factors. Diameter D may also be selected according to remediation requirements and height H. Diameter D and height H may be selected to maximize the surface area of the tube that is illuminated by the light source. Using diameter D as an example, tube thickness, number of coils, height H, and location of light source being equal, diameter D may be selected such that an upper end of the coiled tube casts no shadow on inner side of the lower end of the tube. Tube diameter, number of coils, location of light source, and diameter D may be configured such that light may reach the outer and inner sides of each coil. This increases photon flux and thereby accelerates the rate of pollutant removal. That is, tube diameter, number of coils, location of light source, and diameter D may be configured to increase the number of photons per second per unit of area that reach the tube.

The tubing may be made of a material selected to be structurally resilient to endure system pressures, transportation, rig-in, and harvesting. The tubing may be made of a material selected to be thermally stable, that is, a material that will not expand or contract or cause stress zones due to thermal cycles. The tubing's material may have a high light transmissivity percentage (for example, greater than 80% in the photosynthetically active radiation (PAR) range) to promote energy efficiency, that is, all light of the desired spectrum passes through the material. The tubing's material may be selected to have a spectrum absorption profile that allows a desired spectrum of light (for example, light of wavelengths greater than 380 nm) to pass therethrough.

### Light Source

The light source may be natural light, for example the sun, or artificial light, for example an LED light. A light source 20, 15c may be located around the sides and/or above the coil 10. The spectrum of light delivered by the light source may be selected to promote periphyton growth. Ultraviolet light is that part of the light spectrum ranging in wavelengths from 10 nm to 400 nm. Ultraviolet light may be harmful to periphyton. Therefore, the light source may emit light only with wavelengths greater than 380 nm. Limiting the spectrum of light emitted by the light source may reduce the amount of energy required to power the light source. Energy reduction via reduction of photon escape (for artificial light) further improves energy efficiency by reducing converted input energy into used energy. Certain spectra of light have greater photosynthetic effectiveness in relation to other wavelengths. By using such a photosynthetically effective spectrum, reaction rates increase thereby increasing pollution uptake. In one embodiment, the delivered light may be limited to one or more of the following ranges: 400-675 nm.

### Housing

The housing may have a concave shaped lower section sized to contain the tube. The housing may have a reflective interior to promote illumination of the tube and maximize photon usage.

The housing may have tapered inner diameter, such as being spherical, pyramidal or cone-shaped toward a base 38. The side wall of the housing may have a wider inner diameter at the top than at the bottom such that it defines the spherical, conical, pyramidal shape. The base 38 of the housing may be flat to allow the coiled tube to rest thereon. The side wall 31 of the housing, which may be angled, may have a reflective coating 33 that reflects light onto the tube. The side walls may be planar members arranged vertically in a polygonal shape. The side wall may open upwards, for example in the direction of the light source, to maximize light collection and focus the light on the tube. The side wall may terminate at a lip 32. In one embodiment, the housing may include a lid 34. In one embodiment, lid 34 may mirror the shape of the side wall or it may be flat. The lid terminates at edge 36.

The housing may have openings 39, for example at the top and bottom of the housing, to allow for connections to auxiliary process equipment such as for example power lines, pumps, etc. For example, tubing 10 may pass through openings in the housing and be affixed to the housing. Tubing ends 12 may be attached to collars 13 that are affixed to the housing. This anchors the ends of the tubing to the housing and stabilizes the tubing, that is, prevents the tubing from moving.

The housing may be made of a non-light-absorbent material. The housing may be made of anodized aluminum sheeting. The housing may be made of engineered film, for example, biaxially-oriented polyethylene terephthalate.

### Support Structure

The tube may be supported by a support structure 15, which may be a vertical structure supported on and possibly secured to the base of the housing. For example, support structure 15 may include a core 15a, which may be a cylinder, multi-faceted surfaced polygon (as illustrated in FIG. 6), or an a frame with vertical members such as pillars or arms. The support structure may be transparent to light to allow light to reach the tube through the support structure. One or more light sources 15c may be on an interior face of one or more of the side walls 31.

The support structure may have support arms 15b extending outwards towards the housing to secure the support structure to the side walls 31 of the housing. The support arms may be planar pillars arranged vertically and extending radially outwardly from support structure core 15a. Support arms 15b may include holes and/or collars 13′, in which the coil may rest. The support arms may be attached to create the helical spacing for the wound tubing. The ends of the arms that may connect to the housing may be shaped to hold the tubing so as to minimize the number and area of any stress points that the arms may create on the tubing. This reduces the potential for deformation, kinking, and clogging of the tubing. Further possible advantages of the optional support arms are described at [0036] and elsewhere in this paper.

### Fluid Flow

The apparatus may include a reservoir 60, which may contain a source fluid. There may be a conduit 62 between the reservoir and the tube to allow fluid communication between the reservoir and the tube. The conduit may be attached to a pump 64. There may be a filter 66, for example on the conduit 62, configured to filter fluid flowing from the reservoir to the tube. The filter may include one or more of a tank, cell, and screen configured to remove large debris that may otherwise damage the other components of the apparatus.

The pump may be configured to deliver a desired pressure selected according to factors including: desired turbulence in the tube selected to maximize pollutant contact with periphyton to increase uptake per unit of power used to power the apparatus; concentration of pollutants in the source fluid before treatment; and desired concentration of pollutants in downstream product after treatment. Measurement and control devices 68 may be installed on and included in the apparatus, for example upstream of the tube, in the tube, downstream of the tube, or a combination thereof, to monitor and control one or more of: flow rate, pressure and temperature.

Fluid within the tube may flow in a direction generally from the base of the apparatus to the top of the apparatus. In other words, fluid may be directed to flow generally upwards. This allows the non-pocketed, gradual slope of the helical coil to allow gasses that evolve or are entrapped in the fluid to flow through the reactor. Some of such gasses may be absorbed into the fluid and metabolized, or reacted out, while some of such gases may be allowed to exit the reactor via coil slope or geometry. Generated gasses can move upwardly in the coil so that, rather than becoming trapped and creating occluding bubbles, gasses can be safely accumulated or released, for example, through a valve. This avoids having sections of the tube become stagnant or dry, and prevents gas from inhibiting uptake of pollutants.

Fluid that has been flowed through the tube, thereby undergoing remediation, may be referred to as treated fluid. Treated fluid may be redirected back to source fluid, or back to the tube to cycle the fluid if adequate remediation is not achieved in a first pass through the apparatus. The conduits may contain valves to control the flow. The valves may be solenoid actuated valves, allowing such valves to be programmed and run by a programmatic logic controller, thereby automating cycles.

### Mechanical Harvesting of Periphyton

In one embodiment, the apparatus may be configured to harvest periphyton by mechanical cleaning of the tube 10. For example, a pig launcher 42 may be configured to launch pig 40 into and through the length of the tube 10 to force the periphyton out of the tube 10 to be harvested. With reference to FIG. 5, the tube 10 may have inlet 12 and outlet 14. The pig launcher may be configured to launch the pig into inlet 12, through tube 10, and out of outlet 14. The pig may be configured to scrape periphyton off of the interior of the tube as the pig may travel along the tube's length, and thereby force detached and loose periphyton out of outlet 14. The valves may be configured to direct periphyton to a biomass retriever 50 for separation and storage, and direct the pig to the pig launcher.

The optional support arms described at [0030] and elsewhere in this paper may support the tube against deformation, kinking, and clogging of the tubing, thereby allowing the tubing to be effectively pigged. Deformation, kinking, and clogging of the tubing may trap the pig midway through tube. Support arms prevent deformation, kinking, and clogging of the tube, and thereby facilitate pigging.

It may be advantageous to configure the pig and the tube such that, after pigging, some periphyton, for example, periphyton located in valleys in the rough inner surface of the tube, may be allowed to remain on the inner surface of the tube. The optional rough inner surface of the tubing described at [0020] and elsewhere in this paper also allows for bulk periphyton crop removal while pigging, without completely stripping the periphyton from tube surface. The remaining periphyton permit periphyton growth and fluid remediation to continue without having to re-seed the reactor. Regrowth of periphyton is promoted by allowing some periphyton to remain in crevices on the tube inner surface after pigging. Conversely, removing all of the periphyton from a tube will require periphyton cells to be reseeded to attach to the tube and thereby increase the amount of time it takes to produce the same volume of periphyton.

In one embodiment, the periphyton are regularly harvested, for example by having the pig repeatedly launched through the tube. Regularly harvesting the periphyton maintains peak metabolic rates of growth and promotes high remediation and oxygenation. Regular harvesting also reduces photo-protective dormant periods in bulk growth, thereby allowing more efficient use of the light in the remediation process and speeding up remediation.

Harvesting may prevent photo-protection and promote continual photosynthesis, among other possible benefits. Older periphyton cells may have slower rates of photosynthesis than relatively younger periphyton cells. Harvesting may be done on a schedule configured to maximize the metabolic growth rate and remediation of the apparatus. Regular harvesting removes old periphyton cells, thereby reducing photo-inhibition and respiration periods.

After exiting the outlet of the tube, treated fluid may pass through a biomass retriever 50, which receives the pig, fluid, and harvested biomass, and may separate them, post pigging. Biomass retriever 50 may include a perforated down-pipe connected to the outlet. The down-pipe may be inside a fluid containment cell that may direct the periphyton into the biomass retriever 50. The biomass retriever may be a sump. As illustrated in FIG. 9, biomass retriever may allow the source fluid, pig, and periphyton to separate post-pigging. The pig may be captured at an end of the down-pipe and may be marshalled by the PLC and valves to relaunch the pig. The PLC may be configured to launch and relaunch the pig at intervals selected according to, for example, loss of remediation rate, differential pressure across the reactor, or a combination of these and other process parameters. Treated fluid may pass through the fluid containment cell, which may be configured to separate the periphyton from the treated fluid such that the periphyton may be directed to, for example, the source fluid for further processing, or to the sump; and the treated fluid may be directed to the source fluid. The sump may be emptied, for example by automated valves, and its contents may be sent to a container for further processing or a filter, for example a filter press, for dehydration. The apparatus may include a controller 74 such as a timer or programmable logic controller (PLC) configured to control the valves based on selected routines. The controller may be a timer configured to control the valves, that is, open and close the valves at desired intervals of time.

Periphyton may have facilitated growth in warmer temperature growth liquids. Thus, there may be a heater 72 and the housing may be insulated. For example, the housing may (i) be clad with solid insulation, (ii) be made of a low-emissivity surface, (iii) be vacuum jacketed, (iv) use other insulation means, or (v) use a combination of such means. There may be a heat exchange to recover heat to fluid entering at inlet 12.

The apparatus 100 may include a temperature control device 70 (for example, a heater, a heat sink, or both), configured to regulate the temperature of the fluid. The controller may be configured to control the temperature control device. In one embodiment, the temperature control device may maintain the temperature below 35° C. or 25° C.

### Method

The invention provides a method for phytoremediation reaction. The method includes flowing a fluid through a tube. The fluid may include periphyton and water. The tube may be transparent. Illuminating the tube may promote periphyton growth on the inner surface of the tube. Heating the tube may also promote periphyton growth. Flowing fluid through the tube may allow the fluid to be in fluid communication with the periphyton, and cause the fluid to undergo phytoremediation. The periphyton may be harvested from the tube. Harvesting may include pigging the tube.

### Clauses

Clause 1. A rapid phytoremediation reactor apparatus comprising: a tube, the tube's interior having a rough texture configured for supporting growth of algae.

Clause 2. The apparatus of any one or more of clauses 1-17, wherein the tube is adapted to contain a fluid including algae and water.

Clause 3. The apparatus of any one or more of clauses 1-17, wherein the tube is penetrable to light.

Clause 4. The apparatus of any one or more of clauses 1-17, wherein the tube is substantially continuous.

Clause 5. The apparatus of any one or more of clauses 1-17, wherein the algae is periphyton.

Clause 6. The apparatus of any one or more of clauses 1-17, wherein the tube is coiled.

Clause 7. The apparatus of any one or more of clauses 1-17, wherein the tube is coiled around itself on a plane.

Clause 8. The apparatus of any one or more of clauses 1-17, wherein the tube is coiled vertically into a cylinder shape.

Clause 9. The apparatus of any one or more of clauses 1-17, further comprising a housing configured to contain the tube, the housing including a reflective coating adapted to reflect light into the tube.

Clause 10. The apparatus of any one or more of clauses 1-17, wherein the housing has a base and a cone-shaped side wall, and the tube rests on the base.

Clause 11. The apparatus of any one or more of clauses 1-17, wherein the housing has a top wall that meets the side wall at a lip.

Clause 12. The apparatus of any one or more of clauses 1-17, further comprising a pig launcher and a pig, the pig launcher configured to launch the pig into the tube to scrape algae from the tube for harvesting.

Clause 13. The apparatus of any one or more of clauses 1-17, further comprising a heater configured to heat the tube and its contents.

Clause 14. The apparatus of any one or more of clauses 1-17, further comprising a light source.

Clause 15. A method for cultivating algae, comprising: flowing a liquid through a tube with periphyton growing on an inner surface of the tube, the tube being penetrable to light; illuminating the tube to grow the periphyton on the inner surface of the tube; and harvesting the periphyton.

Clause 16. The method of any one or more of clauses 1-17, wherein harvesting includes pigging the tube.

Clause 17. The method of any one or more of clauses 1-17, wherein flowing the liquid is for phytoremediation of the liquid.

The previous description of the disclosed embodiments is provided to enable any person skilled in the art to make or use the present invention. Various modifications to those embodiments will be readily apparent to those skilled in the art, and the generic principles defined herein may be applied to other embodiments without departing from the spirit or scope of the invention. Thus, the present invention is not intended to be limited to the embodiments shown herein, but is to be accorded the full scope consistent with the claims, wherein reference to an element in the singular, such as by use of the article “a” or “an” is not intended to mean “one and only one” unless specifically so stated, but rather “one or more”. All structural and functional equivalents to the elements of the various embodiments described throughout the disclosure that are known or later come to be known to those of ordinary skill in the art are intended to be encompassed by the elements of the claims. Moreover, nothing disclosed herein is intended to be dedicated to the public regardless of whether such disclosure is explicitly recited in the claims. No claim element is to be construed under the provisions of 35 USC 112, sixth paragraph, unless the element is expressly recited using the phrase “means for” or “step for”.

1. A rapid phytoremediation reactor apparatus comprising: a tube, the tube's interior having a rough texture configured for supporting growth of algae.

2. The apparatus of claim 1, wherein the tube is adapted to contain a fluid including algae and water.

3. The apparatus of claim 1, wherein the tube is penetrable to light.

4. The apparatus of claim 1, wherein the tube is substantially continuous.

5. The apparatus of claim 1, wherein the algae is periphyton.

6. The apparatus of claim 1, wherein the tube is coiled.

7. The apparatus of claim 1, wherein the tube is coiled around itself on a plane.

8. The apparatus of claim 1, wherein the tube is coiled vertically into a cylinder shape.

9. The apparatus of claim 1, further comprising a housing configured to contain the tube, the housing including a reflective coating adapted to reflect light into the tube.

10. The apparatus of claim 9, wherein
the housing has a base and a cone-shaped side wall, and
the tube rests on the base.

11. The apparatus of claim 10 wherein the housing has a top wall that meets the side wall at a lip.

12. The apparatus of claim 1, further comprising a pig launcher and a pig, the pig launcher configured to launch the pig into the tube to scrape algae from the tube for harvesting.

13. The apparatus of claim 1, further comprising a heater configured to heat the tube and its contents.

14. The apparatus of claim 1, further comprising a light source.

15. A method for cultivating algae, comprising:
flowing a liquid through a tube with periphyton growing on an inner surface of the tube, the tube being penetrable to light;
illuminating the tube to grow the periphyton on the inner surface of the tube; and
harvesting the periphyton.

16. The method of claim 15, wherein harvesting includes pigging the tube.

17. The method of claim 15, wherein flowing the liquid is for phytoremediation of the liquid.
